(19) United States
(12) Patent Application Publication
(10) Pub. No.: US 2018/0125667 A1
(43) Pub. Date: May 10, 2018

(54) PROSTHETIC APPARATUS AND SYSTEMS FOR TOTAL KNEE ARTHROPLASTY

(71) Applicant: John Bodeker Savage, Potsdam, NY (US)

(72) Inventor: John Bodeker Savage, Potsdam, NY (US)

(21) Appl. No.: 15/345,291

(22) Filed: Nov. 7, 2016

(51) Int. Cl. A61F 2/38

(52) U.S. Cl. CPC A61F 2/3854; A61F 2/3859; A61F 2002/30604; A61F 2/3886; A61F 2/389

(57) ABSTRACT

Disclosed are various apparatus and systems by which a ball and socket Joint s interposed between prosthetic medial and lateral femorotibial articulations, permitting multidirectional movement and rotation as well as weight-bearing characteristics. Some embodiments have fixed components, while others are modular or quasi-modular in nature. Modifications to traditional designs for embedding a prosthetic tibial component into a tibia are also taught.

### CROSS-REFERENCES TO RELATED APPLICATIONS

Not applicable.

### STATEMENT REGARDING FEDERALLY SPONSORED RESEARCH OR DEVELOPMENT.

Not applicable.

### MICROFICHE APPENDIX:

Not applicable.

### DESCRIPTION

### 1. Field of Technology

At least some embodiments disclosed herein relate, in general, to the field of prosthetic apparatus and systems for bone and joint surgery, including, but not limited to, orthopaedic apparatus and systems for use in total knee arthroplasty.

Background

Total knee arthroplasty (TKA), also referred to as total knee replacement, is a surgical procedure to replace weight-bearing surfaces of the knee joint. The primary indication for TKA is relief of significant, disabling pain caused by severe arthritis or injury.

Design began on the first total knee prosthesis in 1968 in the Biomechanics Unit at the Department of Mechanical Engineering at Imperial College, London. The device, known as the Freeman-Swanson prosthesis, was intended to resurface the whole of the tibiofemoral joint with a polyethylene tibial component articulating with a metallic femoral component, both of which were to be attached to bone with the aid of polymethylmethacrylate cement, and preserve the cruciate ligaments. This condylar prosthesis was first implanted at the London Hospital in 1969 but ultimately proved unreliable. Complications included an unacceptably high incidence of tibial component migration and loosening, an unacceptable incident of patellar pain, wear of the polyethylene component, and an inability reliably to align and stabilize the knee in extension.

The condylar knee was developed independently in the United States in the early 1970s, and a condylar knee with a posterior cruciate-sacrificing design introduced to correct severe knee deformities. By 1974, the standard practice had become replacing the patellofemoral joint and either preserving or sacrificing the cruciate ligaments. Later, condylar knee designs were improved to include non-cemented fixation and modularity using universal instrumentation.

Currently, total knee prostheses still consist of three main components: a femoral component, a tibial component, and a polyethylene spacer serving as an articulating surface for the femoral component. Variations include fixed bearing prostheses, medial pivot devices, apparatus with a rotating platform and mobile bearing, posterior cruciate ligament (PCL)-retaining prostheses, and PCL-substituting designs. The type of prosthesis selected by a surgeon may be based on several factors, including, but not limited to, the particular anatomy of a given patient, the degree of knee deformity present, ligament stability, and the quality of soft tissues.

Fixed-bearing (FB) knee designs contemplate fixing one or more plastic spacers securely on the underlying tibial component, whereas rotating-platform (RP) knee prostheses allow rotation of a plastic spacer on the tibial component. Theoretically, allowing rotation of a plastic spacer helps to replicate the natural limited rotation of a normal knee when extended fully, and reduces the shear stress on the articulating surface caused by the motion of the femoral component.

RP knee prostheses, however, are more dependent on the surrounding ligaments and soft tissues for to prevent dislocation. Additionally, they may be prone to more frequent soft tissue impingement. Their use tends to be restricted to younger, more active patients. The life of a RP knee is comparable to that of an FB knee.

The medial pivot (MP) knee attempts to recreate typical knee motion in which the medial femoral condyle remains relatively stationary on the tibia during knee flexion, as opposed to the lateral femoral condyle, which translates during flexion. This design has a closer fitting articulation between medial femoral and tibial components, which tends to limit motion more than other knee prosthetic designs.

Some surgeons routinely remove the posterior cruciate ligament, and will use posterior-stabilized (PS) components; however, if the posterior cruciate ligament is in good condition, some surgeons will retain this ligament and use cruciate retaining (CR) components instead. PS knees allow for the reliable restoration of knee kinematics, small improvements in motion compared to CR knees, and the theoretical reduction in polyethylene wear. PS knees are specifically indicated in patients with severe deformity, severe flexion contracture, previous removal of the knee cap, and during revision surgery. By contrast, CR knees require less bone to be removed, decrease the chance of a “patellar clunk” syndrome—a sometimes painful condition caused by growth of interposing soft tissue, typically at the superior pole of the patella, associated with a mechanical catching or clunking during active extension following TKA—and decrease the potential complications associated with having a polyethylene post, such as fracture of the post.

Common to all of these existing prosthetic knee designs is an effort to model the implant on the natural anatomy of the knee as closely as possible so as to mimic the kinematics of a normal knee. A knee design which changes the natural architecture of the joint could provide greater mobility and lateral stability than current TKA implants, which more closely imitate the shape of the base of the femur and its intersection with the top of the tibia.

### BRIEF DESCRIPTION OF THE SEVERAL VIEWS OF THE DRAWINGS

The embodiments illustrated are by way of example, and not limitation, in the figures of the accompanying drawings in which like references indicate similar elements.

FIG. 1 is an exploded view of an embodiment, in relation to the lower extremity of a femur and the upper extremity of a tibia, with a modular trunnion and tibial ball and a single horseshoe-style insert,

FIG. 2 is an elevated posterior view of an embodiment revealing a trunnion inserted into a tibial trunnion post of a tibial component and a tibial ball to be received by the trunnion.

FIG. 3 is an elevated anterior view of an embodiment with a trunnion inserted into a tibial trunnion post of a tibial component, a tibial ball that has been received by the trunnion, and a femoral socket of a femoral component that has received the tibial ball.

FIG. 4 is an elevated posterior view of an embodiment with a trunnion inserted into a tibial trunnion post of a tibial component, a tibial ball that has been received by the trunnion, and a femoral socket of a femoral component that has received the tibial ball.

FIG. 5 is a top plan view of an embodiment with a trunnion and tibial ball of a tibial component that has been received by a femoral socket in a femoral component.

FIG. 6 is a bottom plan view of an embodiment with a trunnion and tibial ball of a tibial component that has been received by a femoral socket of a femoral component.

FIG. 7A is a side elevational view of an embodiment with a trunnion inserted into a tibial trunnion post of a tibial component, and a tibial ball that has been received by the trunnion, with prosthetic femoral condyles and a femoral socket of a femoral component as positioned upon full extension of a patient's leg.

FIG. 7B is a side elevational view of a trunnion inserted into a tibial trunnion post of a tibial component of a tibial component, and a tibial ball that has been received by the trunnion, with prosthetic femoral condyles and a femoral socket of a femoral component as positioned upon a 45° flexion of a patient's leg.

FIG. 7C is a side elevational view of an embodiment with a trunnion inserted into a tibial trunnion post of a tibial component, and a tibial ball that has been received by the trunnion, with prosthetic femoral condyles and a femoral socket of a femoral component as positioned upon a 90° flexion of a patient's leg.

FIG. 8A is a cross-sectional view of an embodiment with a trunnion inserted into a tibial trunnion post of a tibial component, and a tibial ball that has been received by the trunnion, with prosthetic femoral condyle and femoral socket of a femoral component as positioned upon full extension of a patient's leg.

FIG. 8B is a cross-sectional view of an embodiment with a trunnion inserted into a tibial trunnion post of a tibial component, and a tibial ball that has been received by the trunnion, with prosthetic femoral condyle and femoral socket of a femoral component as positioned upon full a 45° flexion of a patient's leg.

FIG. 8C is a cross-sectional view of an embodiment with a trunnion inserted into a tibial trunnion post of a tibial component, and a tibial ball that has been received by the trunnion, with prosthetic femoral condyle and femoral socket of a femoral component as positioned upon a 90° flexion of a patient's leg.

FIG. 9 is an exploded view of an embodiment, in relation to the lower extremity of a femur and the upper extremity of a tibia, with a trunnion and ball integrated into a tibial component, and with bilateral inserts.

FIG. 10 is an elevated anterior view of an embodiment with a trunnion and ball integrated into a tibial component, and a femoral socket of a femoral component that has received the ball.

FIG. 11 is an elevated posterior view of an embodiment with a trunnion and ball, integrated into a tibial component, wherein the ball has been received by a femoral socket of a femoral component that has received the ball.

FIG. 12A is a side elevational view of an embodiment with a trunnion and a ball that has been integrated into a tibial component, and with prosthetic femoral condyles and a femoral socket of a femoral component as positioned upon full extension of a patient's leg.

FIG. 12B is a side elevational view of an embodiment with a trunnion and a ball that has been integrated into a tibial component, and with prosthetic femoral condyles and a femoral socket of a femoral component as positioned upon full a 45° flexion of a patient's leg.

FIG. 12C is a side elevational view of an embodiment with a trunnion and a ball that has been integrated into a tibial component, and with prosthetic femoral condyles and a femoral socket of a femoral component as positioned upon a 90° flexion of a patient's leg.

FIG. 13A is a cross-sectional view of an embodiment with a trunnion and a ball that has been integrated into a tibial component, and with prosthetic femoral condyles and a femoral socket of a femoral component as positioned upon full extension of a patient's leg.

FIG. 13B is a cross-sectional view of an embodiment with a trunnion and a ball that has been integrated into a tibial component, and with prosthetic femoral condyles and a femoral socket of a femoral component as positioned upon a 45° flexion of a patient's leg.

FIG. 13C is a cross-sectional view of an embodiment with a trunnion and a ball that has been integrated into a tibial component, and with prosthetic femoral condyles and a femoral socket of a femoral component as positioned upon a 90° flexion of a patient's leg.

FIG. 14 is an orthogonal view of the anterior of an embodiment with a horseshoe-style insert placed over a tibial platform of a tibial component embedded in a tibia, further reflecting a trunnion inserted into a tibial trunnion post, and a ball that has been received by the trunnion.

FIG. 15 is an orthogonal view of the anterior of an embodiment with bilateral inserts placed over a tibial platform of a tibial component embedded in a tibia, further reflecting a trunnion and a ball integrated into a tibial component.

FIG. 16 is an orthogonal view of the anterior of an embodiment with bilateral inserts placed over a tibial platform of a tibial component embedded in a tibia, reflecting a trunnion and a ball integrated into a tibial component, and a femoral component embedded in a femur with a femoral socket that has received the ball and prosthetic condyles of said femoral component that have been received by the articulating inserts.

FIG. 17 is an exploded view of an embodiment, in relation to the lower extremity of a femur and the upper extremity of a tibia, with a ball integrated into a femoral component between prosthetic condyles, a tibial socket integrated into the tibial platform of a tibial component for receiving the ball, and bilateral inserts to accept the prosthetic condyles.

FIG. 18 is an elevated anterior view of an embodiment with a ball integrated into a femoral component between prosthetic condyles, a tibial socket integrated into the tibial platform of a tibial component for receiving the ball, and bilateral inserts to accept the prosthetic condyles.

FIG. 19 is an elevated posterior view of an embodiment with a ball integrated into a femoral component between prosthetic condyles, a tibial socket integrated into the tibial platform of a tibial component for receiving the ball, and bilateral inserts to accept the prosthetic condyles.

FIG. 20 is a top plan view of an embodiment with a ball integrated into a femoral component between prosthetic condyles, a tibial socket integrated into the tibial platform of a tibial component for receiving the ball, and bilateral inserts to accept the prosthetic condyles.

FIG. 21 is a bottom plan view of an embodiment with a ball integrated into a femoral component between prosthetic condyles, a tibial socket integrated into the tibial platform of a tibial component for receiving the ball, and bilateral inserts to accept the prosthetic condyles.

FIG. 22A is a side elevational view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon full extension of a patient's leg.

FIG. 22B is a side elevational view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon a 45° flexion of a patient's leg.

FIG. 22C is a side elevational view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon a 90° flexion of a patient's leg.

FIG. 23A is a cross-sectional view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon full extension of a patient's leg.

FIG. 23B is a cross-sectional view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon a 45° flexion of a patient's leg.

FIG. 23C is a cross-sectional view of an embodiment with a ball integrated into a femoral component between prosthetic condyles, a tibial socket integrated into the tibial platform of a tibial component for receiving the ball, and bilateral inserts to accept the prosthetic condyles, as the prosthetic condyles are positioned upon a 90° flexion of a patient's leg.

FIG. 24 is an cutaway orthogonal view of an embodiment with a tibial component having a tusk-shaped tibial anchor and an integrated trunnion with one of two reaming slots exposed.

FIG. 25 is a cutaway anterior view of an embodiment with a tibial component having a tusk-shaped tibial anchor and an integrated trunnion with each of two reaming slots exposed.

FIG. 26 is a cross-sectional view of an embodiment with a trunnion and a tusk-shaped tibial anchor integrated into a tibial component, and a tibial ball that has been received by the trunnion.

REFERENCE NUMERALS IN THE DRAWINGS

| | | | |
|---|---|---|---|
| 102 | System | 104 | Lower extremity of a femur |
| 104A | Lateral epicondyle | 104B | Medial epicondyle |
| 104C | Intercondylar fossa | 106 | Upper extremity of a tibia |
| 106A | Tibial lateral condyle | 106B | Tibial medial condyle |
| 108 | Femoral component | 110 | End of a femur |
| 112 | Tibial component | 114 | End of a tibia |
| 116 | Integrated femoral socket | 118 | Outer surface of femoral socket |
| 120 | Inner surface of femoral socket | 122 | Prosthetic condyle |
| 122A | Condylar protrusion | 124 | Joinder element |
| 126 | Tibial ball | 128 | Femoral socket liner |
| 130 | Modular trunnion | 130A | Proximal end of modular trunnion |
| 130B | Distal end of modular trunnion | 132 | Trunnion post |
| 134 | Tibial platform | 136 | Tibial anchor |
| 138 | Tibial anchor post | 140 | Tibial anchor wing |
| 142 | Anchor posthole | 144 | Tibial wing slot |
| 146 | Trunnion posthole | 148 | Horseshoe-style insert |
| 150 | Lip | 202 | Tibial ball cavity |
| 204 | Insertion end of anchor post | 206 | Femoral post |
| 208 | Posterior flange | 902 | System |
| 904 | Integrated trunnion and ball | 906 | Bilateral insert |
| 1702 | System | 1704 | Femoral ball |
| 1706 | Tibial socket | 1708 | Femoral cup |
| 1710 | Tibial socket liner | 2402 | Tibial stem |
| 2404 | Integrated tibial trunnion | 2406 | Reaming slot |
| 2408 | System | | |

### DETAILED DESCRIPTION OF THE INVENTION

The following description and drawings are illustrative and are not to be construed as limiting. Numerous specific details are described to provide a thorough understanding. However, in certain instances, well-known or conventional details are not described in order to avoid obscuring the description. References to “one embodiment” or “an embodiment” in the present disclosure are not necessarily references to the same embodiment; and, such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” or substantially similar phrases in various places in the specification are not necessarily all referring to the same embodiment, nor are separate or alternative embodiments mutually exclusive of other embodiments. Moreover,various features are described which may be exhibited by some embodiments and not by others. Similarly, various requirements are described which may be requirements for some embodiments but not for other embodiments.

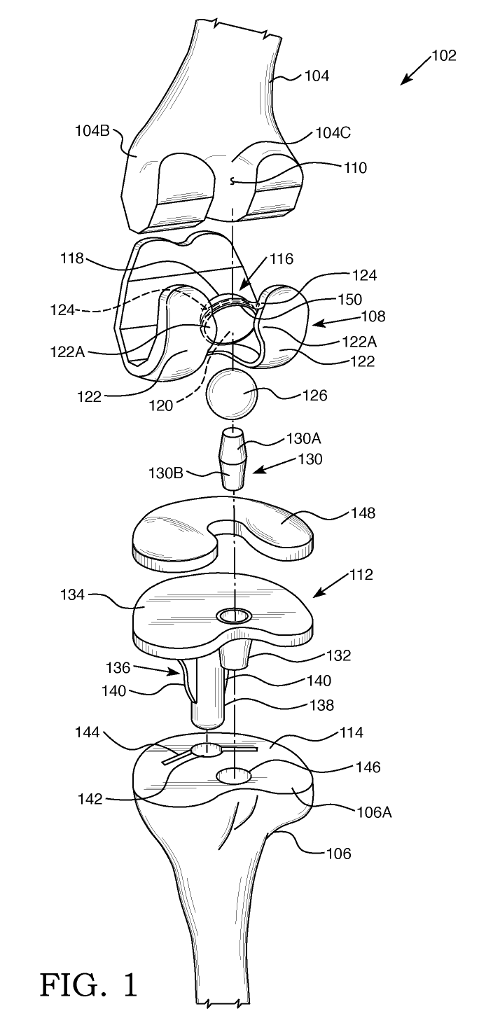

FIG. 1 presents an exploded view of al embodiment of a system 102 in relation to the lower extremity of a femur 104 and the upper extremity of a tibia 106, each of which has been prepared for implantation of the embodiment through the resection of damaged cartilage surfaces along with a small amount of underlying bone. In an embodiment, a femoral component 108 may be configured to be accepted by the prepared surface of the end of the femur 110, including the lateral epicondyle 104A, the medial epicondyle 104B, and the intercondylar fossa 104C, and a tibial component 112 configured to be accepted by the prepared surface of the end of the tibia 114, including the tibial lateral condyle 106A and the tibial medial condyle 106B. In various embodiments, a femoral component 108 and a tibial component 112 may be comprised of assorted types of stainless steel, titanium, titanium alloys, cast or wrought cobalt, or other biocompatible materials, alone or in combination, currently known in the art. Femoral components and tibial components will be sized to fit according to the anatomy of a given patient

In an embodiment, a femoral component 108 may have an integrated femoral socket 116 with an outer surface 118 and an inner surface 120, said femoral socket located between prosthetic condyles 122 of the femoral component 108 and attached to the femoral component 108 by one or more joinder elements 124. A femoral socket 116 may be configured to accept a tibial ball 126 in an embodiment and provide an articulating surface for the tibial ball 126. Some embodiments may have prosthetic condyles 122 with inner condylar protrusions 1.22A.

Some embodiments may comprise a femoral socket 116 with an inner surface 120 of metal and a tibial ball 126 of polyethylene. In other embodiments, a. liner 128 of polyethylene or ceramic material may be secured to the inner surface 120 of a femoral socket 116 to accept a tibial ball 126 made of metal or ceramic material. In various embodiments, other combinations of materials known in the art may be used to create an articulation between a tibial ball 126 and the inner surface 120 of a femoral socket.

A tibial ball 126 may be configured in an embodiment to engage a modular trunnion 130 with a proximal end 130A and a distal end 130 B, said modular trunnion 130 adapted to be inserted into a hollow trunnion post 132 of a tibial component 112. In various embodiments, the proximal end 130A or the distal end 1308 of a modular trunnion 130, or both, may be tapered.

In an embodiment, a tibial component 112 includes a tibial platform 134 and a tibial anchor 136 comprised of a tibial anchor post 138 and twin tibial anchor wings 140 on either side of the tibial anchor post 138. A tibial anchor 136 may be joined to the upper extremity of a tibia 106 using a posthole 142 and tibial wing slots 144 that have been cut or reamed into the upper extremity of a tibia 106. In embodiments including a hollow trunnion post 132, the insertion of the hollow trunnion post 132 into a trunnion posthole 146 in the upper extremity of a tibia 106 may add additional stability to an implanted tibial component 112. In an embodiment, a hollow trunnion post 132 may be tapered to facilitate insertion into a trunnion posthole 146.

In an embodiment, one or more inserts may be employed to provide one or more articulating surfaces for prosthetic condyles 122 of a femoral component 108. Inserts may be constructed of polyethylene or other suitable materials currently known in the art. As depicted in FIG. 1, a single horse-shoe style insert 148 may be placed over a tibial platform 134.

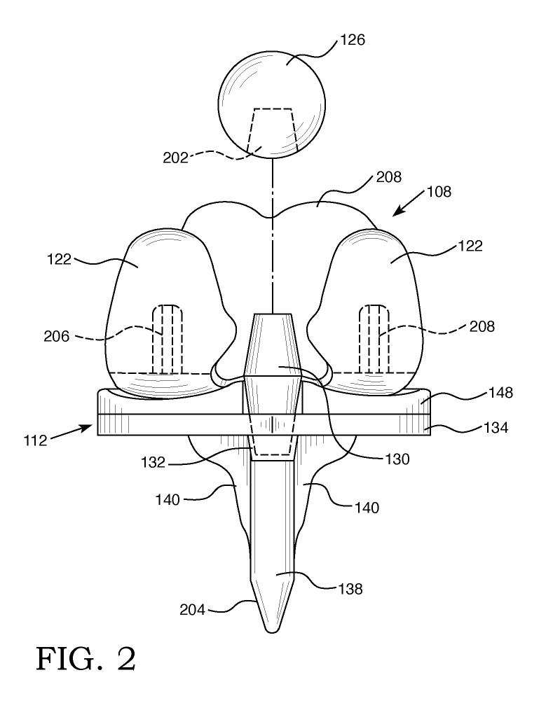

FIG. 2 illustrates in an elevated posterior view an embodiment with a modular tibial ball 126 configured to be received by a modular trunnion 132 at its proximal end 130A via a tibial ball cavity 202, with the distal end 130B of the modular trunnion 132 inserted into a hollow trunnion post 132 of a tibial component 112. In an embodiment, an anchor post 138 may be tapered at its insertion end 204 to facilitate its placement into a tibial posthole 142 in the upper extremity of a tibia 106. FIG. 2 also depicts femoral posts 206 in a femoral component 108 that may be employed to affix the femoral component 108 to the lower extremity of a femur 104 by means of boreholes drilled in the lateral epicondyle 104A and the medial epicondyle 104B, as well as a posterior flange 208 of a femoral component 108.

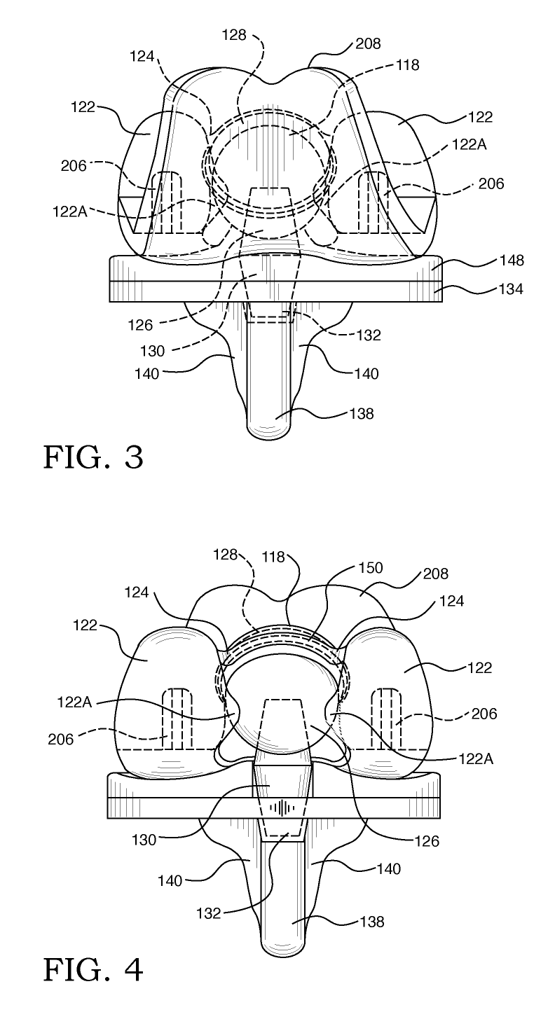

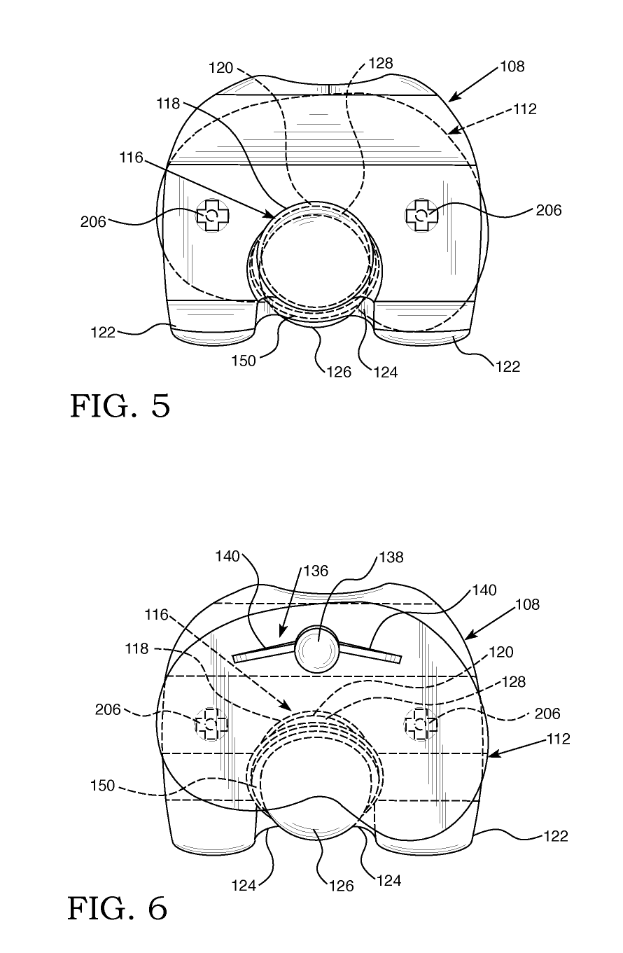

FIG. 3 and FIG. 4 are, respectively, an elevated anterior view and an elevated posterior view of one embodiment with a modular trunnion 130 inserted into a tibial trunnion post 132 of a tibial component 112, a tibial ball 126 that has been received by the modular trunnion 130, and an integrated femoral socket 116 of a femoral component 108 that has received the tibial ball 126. As illustrated in FIG. 4, in an embodiment, an integrated femoral socket 116 may be attached to a femoral component 108 by one or more joinder elements 124 and may have a liner 128 secured to its inner surface 120 against which a tibial ball 126 may articulate. A liner 128 may be cemented or cementlessly secured to the inner surface 120 of a femoral socket 116 in an embodiment, and may have a lip 150 extending around the perimeter of a femoral socket 116 at the juncture of the outer surface 118 and the inner surface 120 of the femoral socket 116, as illustrated in FIG. 4. In FIG. 5 a top plan view is presented, and in FIG. 6 a bottom plan view, of an embodiment with a tibial ball 126 that has been received by a femoral socket 116 of a femoral component 108.

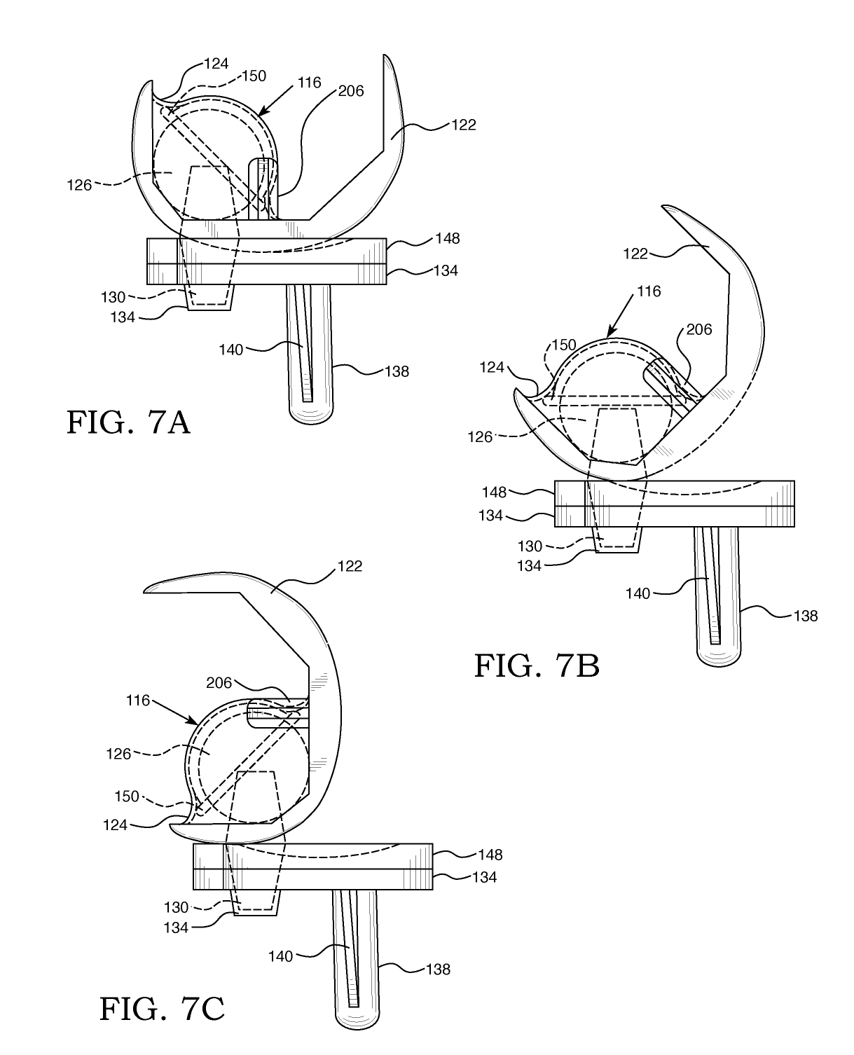

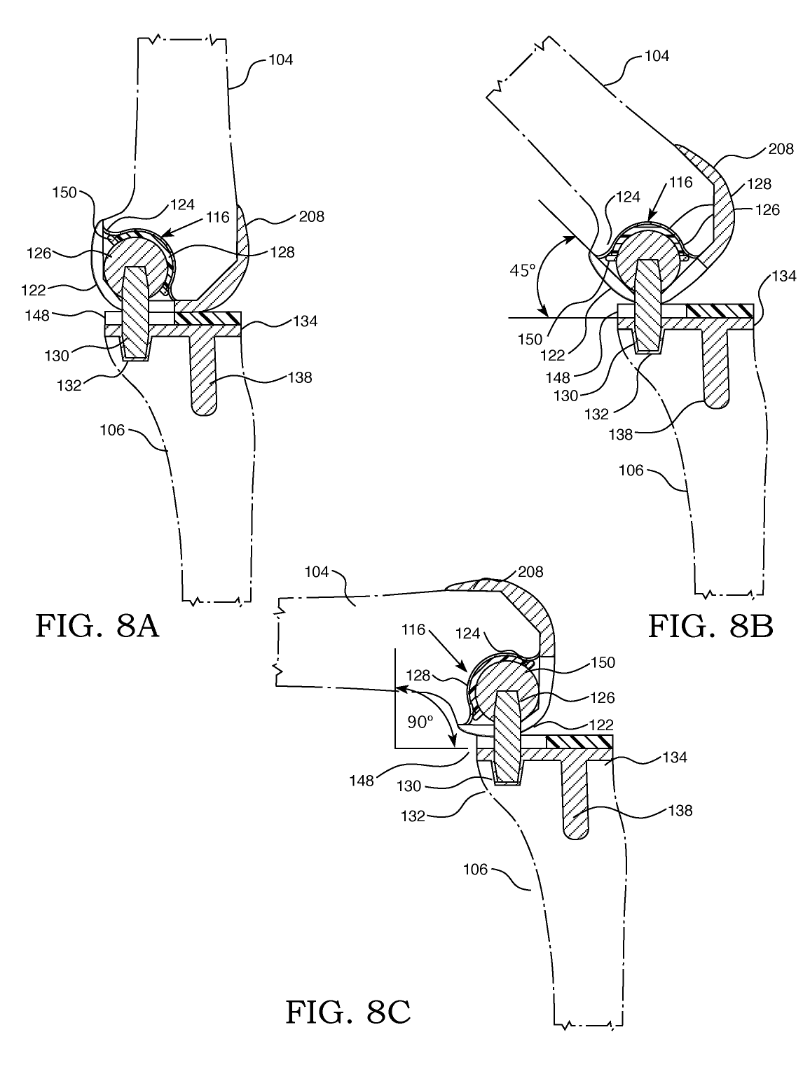

FIG. 7A, FIG. 7B, and FIG. 7C are side elevational views of an embodiment with a modular trunnion 130 inserted into a tibial trunnion post 132 of a tibial component 112, and a tibial ball 126 that has been received by the modular trunnion 130, with prosthetic femoral condyles 122 and a femoral socket 116 of a femoral component 108 as configured to be positioned, respectively, upon full extension of a patient's leg, upon a 45° flexion of a patient's leg, and upon a 90° flexion of a patient's leg. As illustrated by these three views, a tibial ball 126 remains stationary with respect to a modular trunnion 130 and a tibial component 112, while a femoral socket 116 against which the tibial ball 126 articulates is configured to move about the center of the axis of rotation of a femur. Cross-sectional views in FIGS. 8A, 8B, and 8C likewise reflect a femoral socket 116 configured to move progressively about the center of the axis of rotation of a femur from full extension to 90° of flexion.

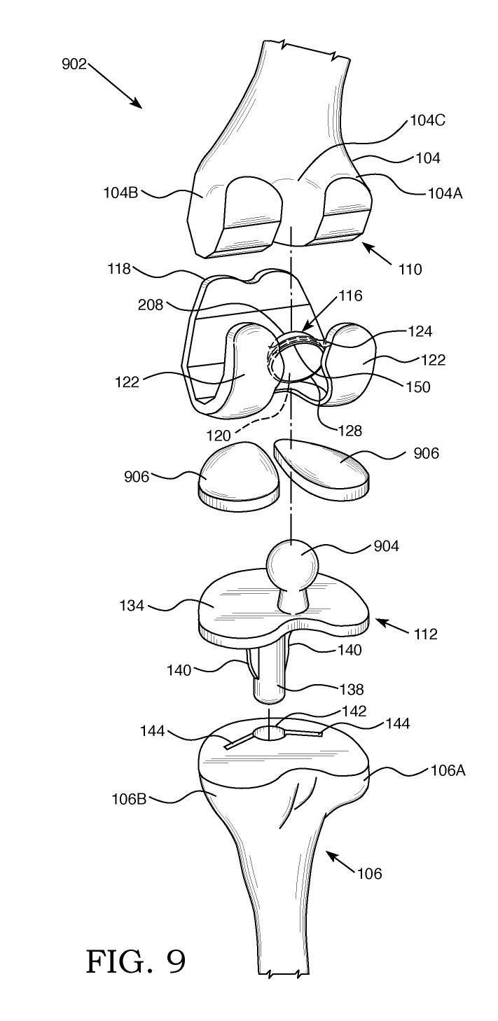

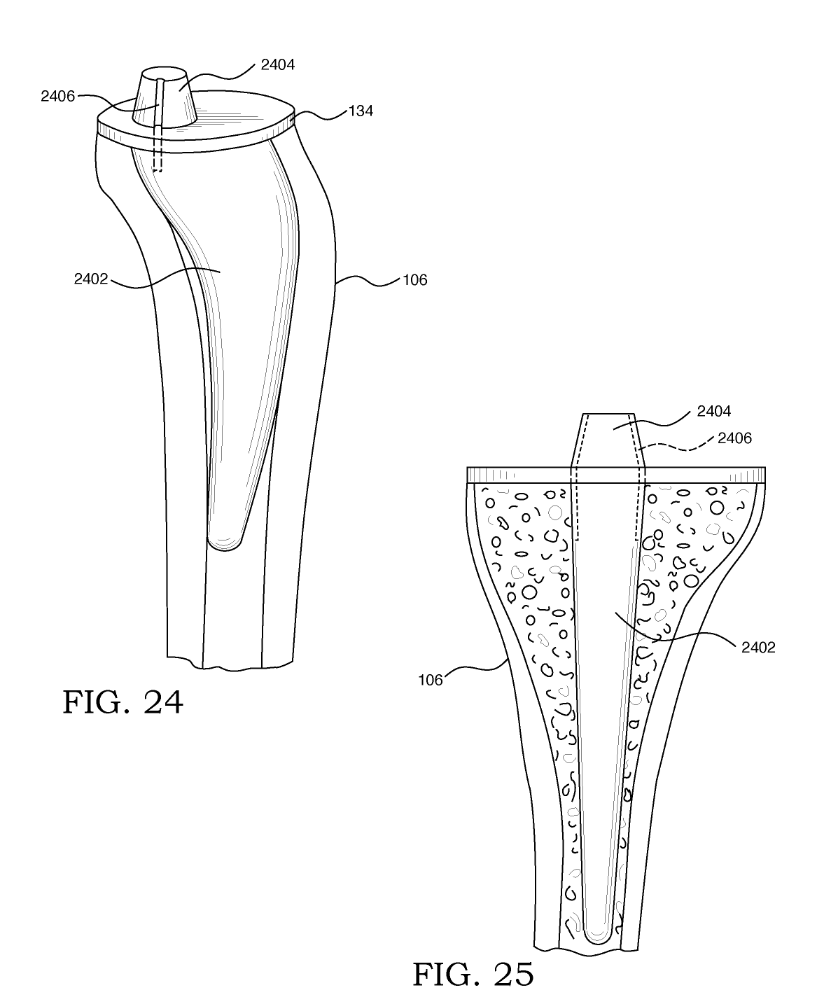

Illustrated in FIG. 9 is an exploded view of an embodiment of a system 902 in relation to the lower extremity of a femur 104 and the upper extremity of a tibia 106, with a tibial component 112 comprising an integrated trunnion and ball 904 at its tibial platform 134. In other embodiments, only an integrated tibial trunnion 2404 (see FIG. 24) would be unified with a tibial platform 134, and a tibial ball 126 could be configured to be accepted by that integrated tibial trunnion 2404. In an embodiment, the ball of an integrated trunnion and ball 904 may be configured to articulate against the inner surface of a femoral socket 120, or against a femoral socket liner 128 embedded in or attached to the inner surface of said femoral socket 120. Likewise, in an embodiment, a tibial ball 126 to be accepted by an integrated tibial trunnion 2404 may be configured to articulate against the inner surface of a femoral socket 120, or against a femoral socket liner 128 embedded in or attached to the inner surface of said femoral socket 120.

Additionally, FIG. 9 displays bilateral inserts 906 for placement over a tibial platform 134 on either side of an integrated trunnion and ball 904 that may be utilized in an embodiment. As with a horse-shoe style insert 148 placed over a tibial platform 134, in an embodiment, bilateral inserts 906 may provide an articulating surface for each prosthetic condyle 122 of a femoral component 108, and these bilateral inserts 906 may be constructed of polyethylene, ceramic, or other suitable materials currently known in the art.

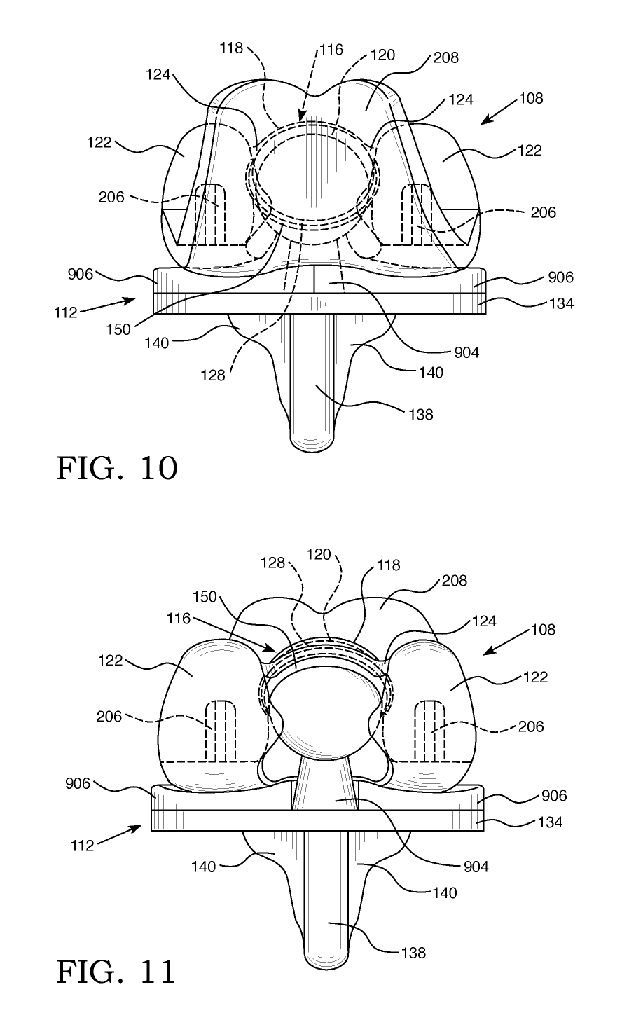

FIG. 10 and FIG. 11, respectively, present an elevated anterior view and an elevated posterior view of an embodiment with an integrated trunnion and ball 904 in a tibial component 112, and a femoral socket 116 of a femoral component 108 that has received the ball of the integrated trunnion and ball 904.

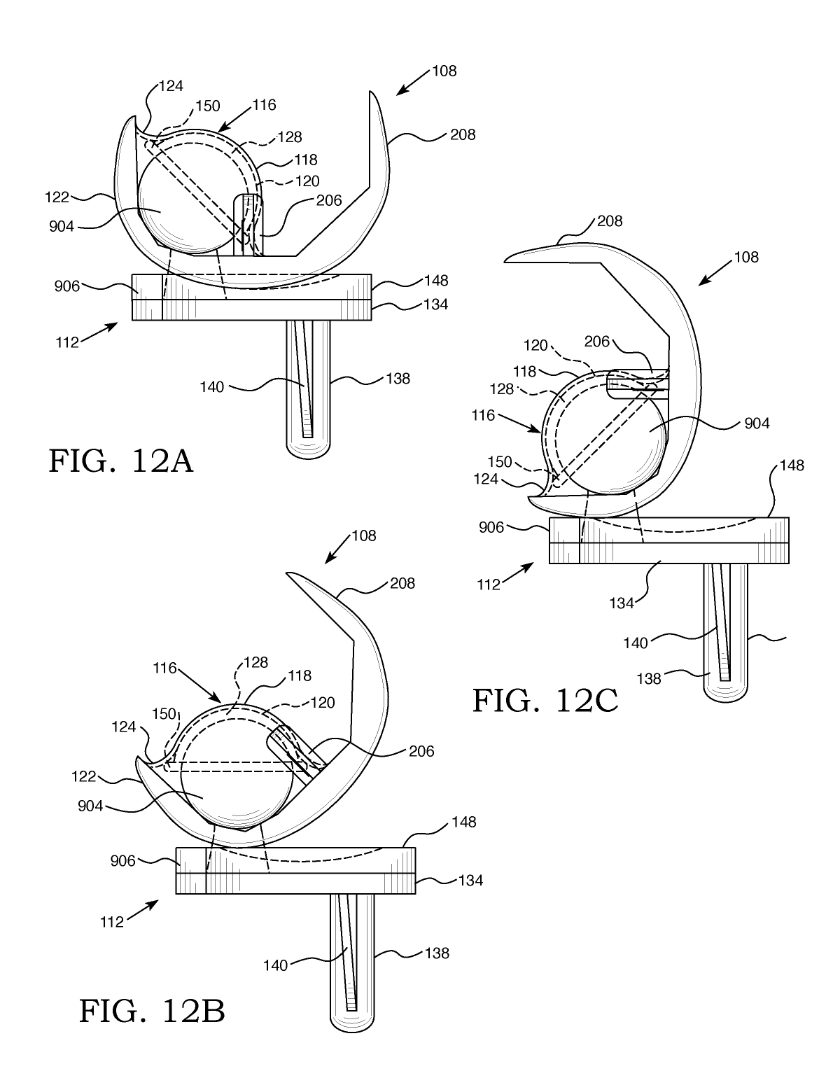

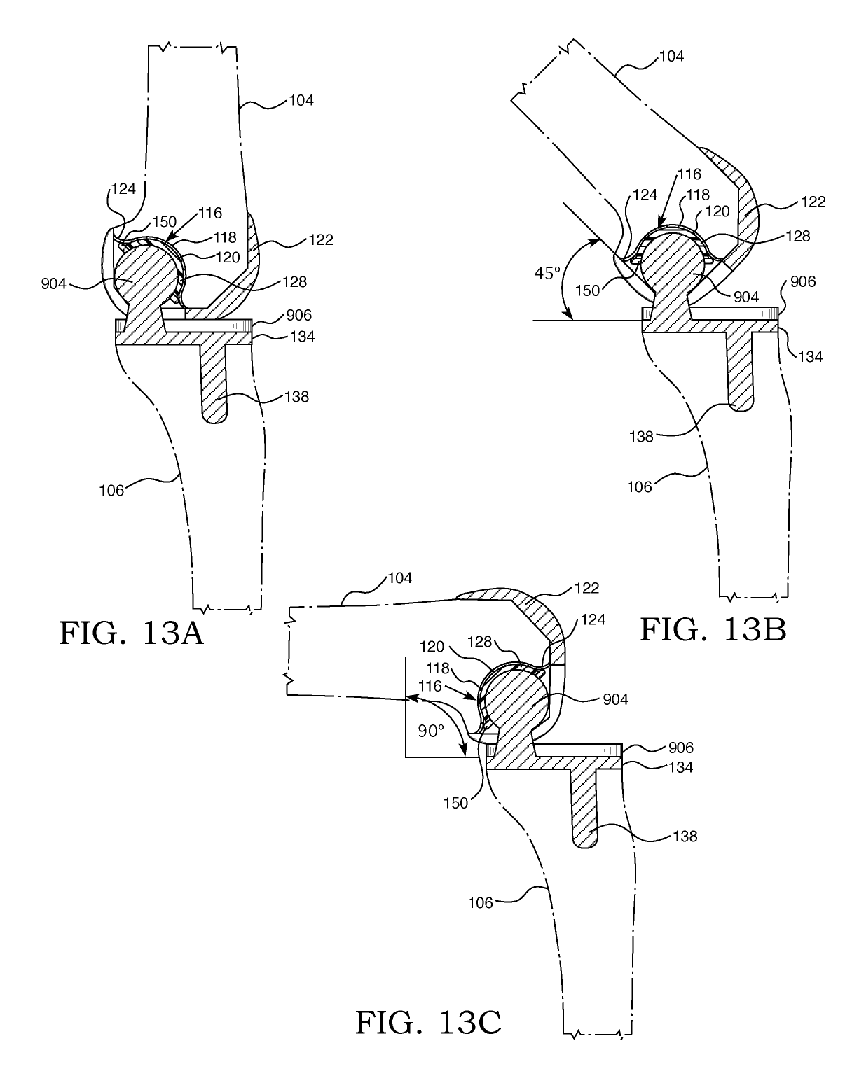

FIG. 12A, FIG. 12B, and FIG. 12C are, respectively, side elevational views of an embodiment with an integrated trunnion and a ball 904 in a tibial component 112, with prosthetic femoral condyles 122 and a femoral socket 116 of a femoral component 108 as configured to be positioned, respectively, upon full extension of a patient's leg, upon a 45° flexion of a patient's leg, and upon a 90° flexion of a patient's leg. FIG. 13A, FIG. 13B, and FIG. 13C display cross-sectional views of an embodiment revealing a femoral socket 116 configured to move progressively about the center of the axis of rotation of a femur from full extension to 90° of flexion.

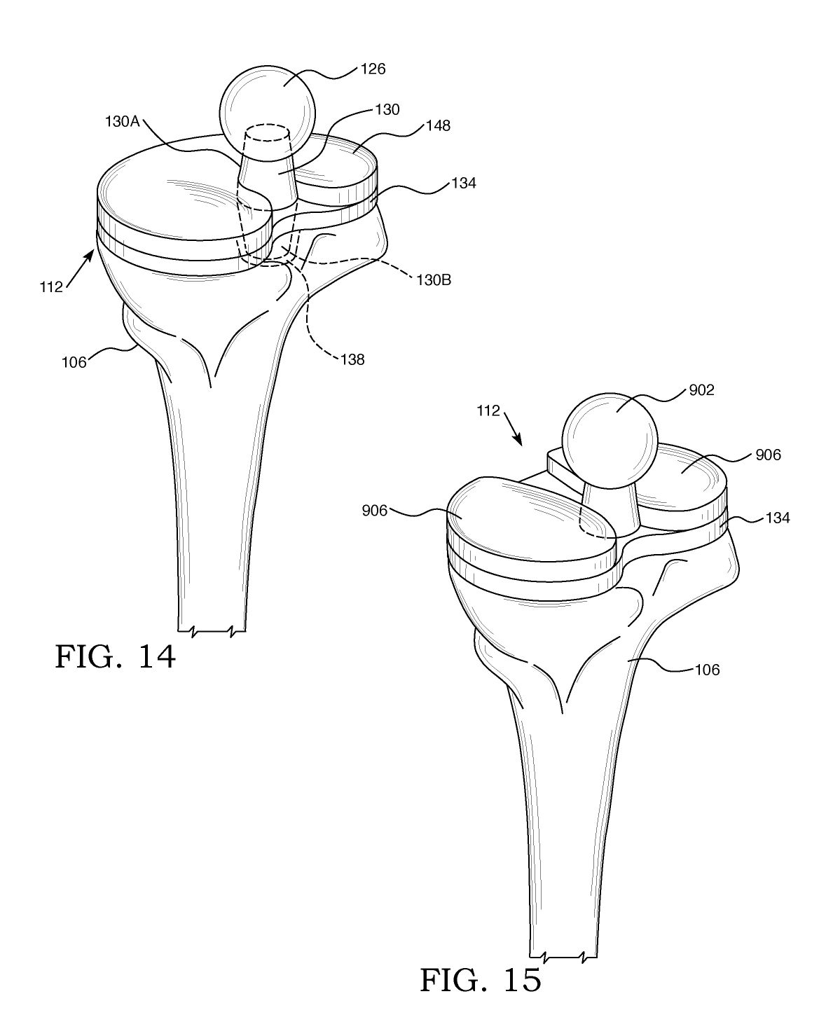

FIG. 14 is an orthogonal view of the anterior of an embodiment with a horseshoe-style insert 148 placed over a tibial platform 134 of a tibial component 112 embedded the upper extremity of a tibia 106, reflecting a modular trunnion 130 inserted into a tibial trunnion post 138, and a ball 126 that has been received by the modular trunnion 130. FIG. 15 is an orthogonal view of the anterior of an embodiment with bilateral inserts 906 placed over a tibial platform 134 of a tibial component 112 embedded in the upper extremity of a tibia 106, further reflecting an integrated trunnion and ball 904 in a tibial component 112.

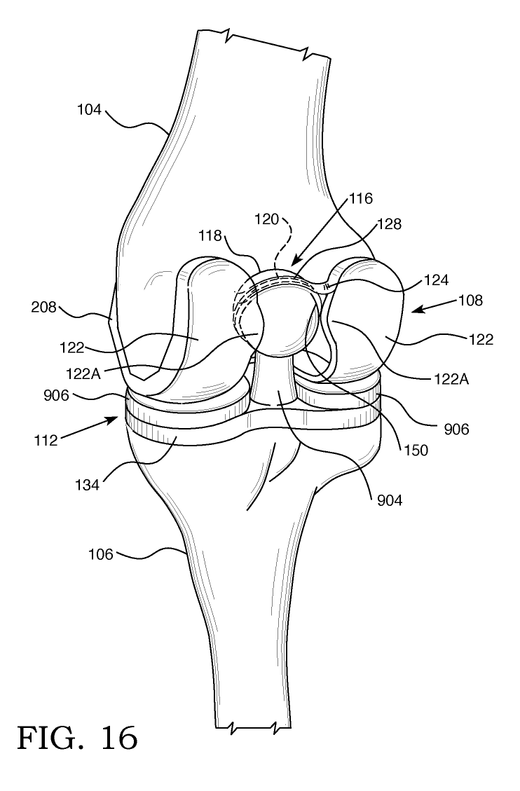

The orthogonal view of the anterior of an embodiment illustrated in FIG. 16 reveals bilateral inserts 906 placed over a tibial platform 134 of a tibial component 112 embedded in the upper extremity of a tibia 106, showing an integrated trunnion and ball 904 in a tibial component 112, and a femoral component 108 embedded in the lower extremity of a femur 104 with a femoral socket 116 that has received the ball of the integrated trunnion and ball 904 and prosthetic condyles 122 of said femoral component 108 that have been received by the bilateral inserts 906.

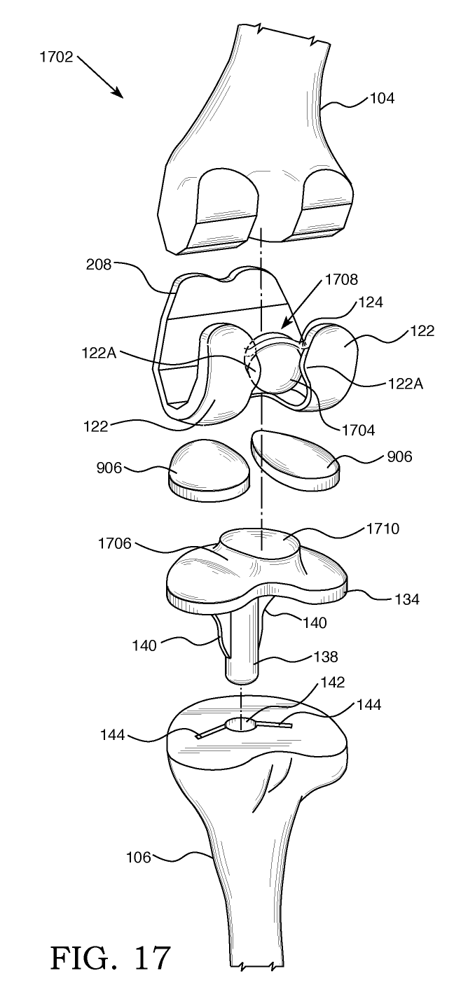

FIG. 17 is an exploded view of an embodiment of a system 1702 in relation to the lower extremity of a femur 104 and the upper extremity of a tibia 106 with a femoral ball 1704 seated in a femoral component 108 between prosthetic condyles 122, a tibial socket 1706 integrated into the tibial platform 134 of a tibial component 112 for receiving the femoral ball 1704 and providing an articulating surface for said femoral ball, and bilateral inserts 906 to accept the prosthetic condyles 122 and provide them with an articulating surface. In an embodiment, a femoral ball 1704 may be affixed inside a femoral cup 1708 integrated into a femoral component by one or more joinder elements 124. A femoral ball 1704 may be configured to rotate freely within a femoral cup 1708 in an embodiment. Inner condylar protrusions 122A on prosthetic condyles 122 may help keep a femoral ball 1704 in place.

An embodiment may utilize a femoral ball 1704 comprised of assorted types of stainless steel, titanium, titanium alloys, cast or wrought cobalt, polyethylene, ceramic, or other biocompatible materials, alone or in combination, currently known it the art. A tibial socket 1706 integrated into the tibial platform 134 of a tibial component 112 for receiving a femoral ball 1704 may, in an embodiment, include a tibial socket liner 1710 of polyethylene, ceramic, or other appropriate materials or combinations of materials currently known in the art, secured to the tibial socket 1706 with cement or cementlessly, to accept a femoral ball 1704 for articulation.

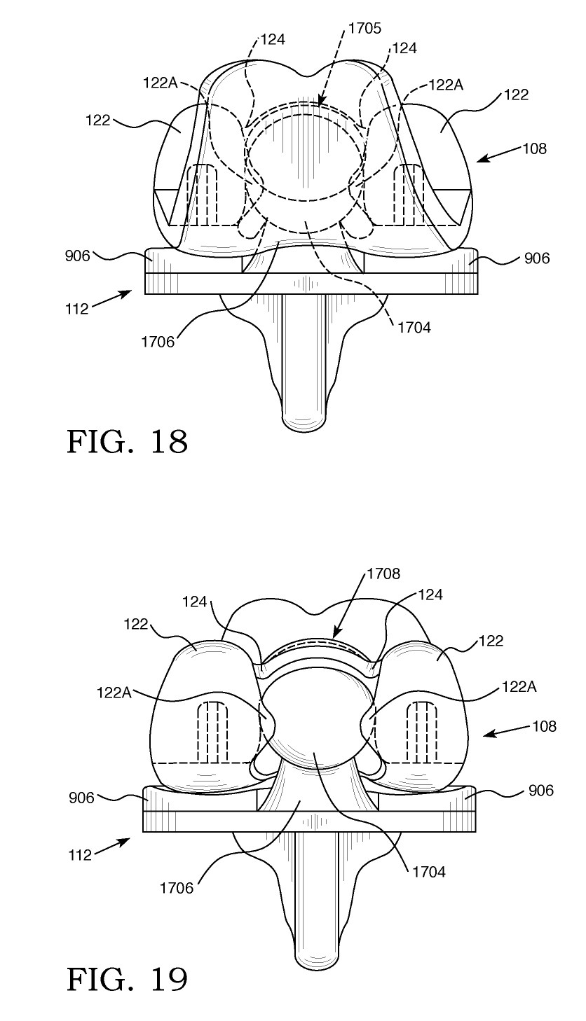

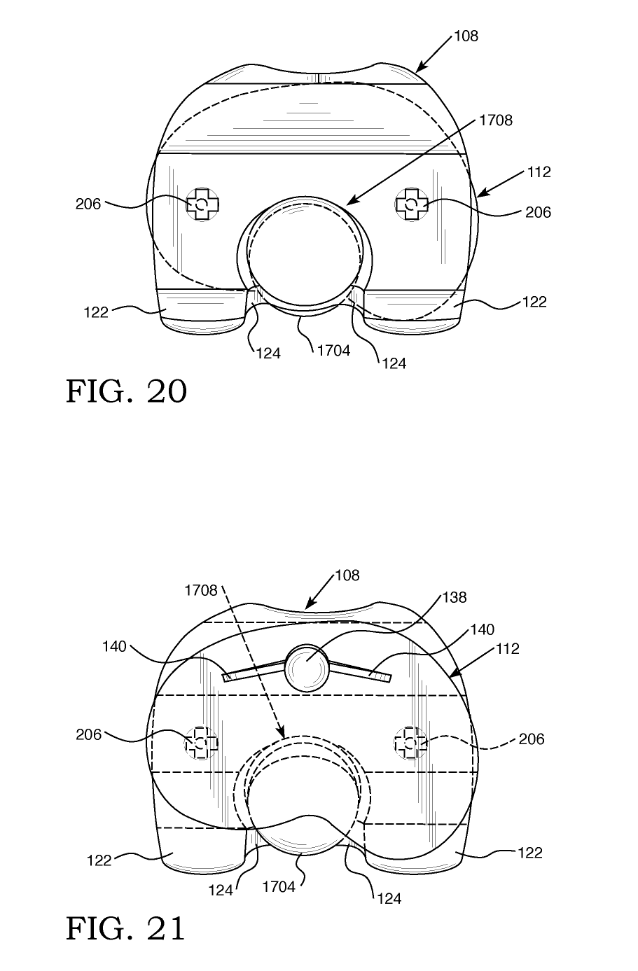

An elevated anterior view and an elevated posterior view of an embodiment are depicted in FIG. 18 and FIG. 19, respectively, with a femoral ball 1704 integrated into a femoral cup 1708 of a femoral component 108 between prosthetic condyles 122 which include condylar protrusions 122A, said femoral ball 1704 seated in a tibial socket 1706 integrated into a tibial platform 134 of a tibial component 112, and bilateral inserts 906 to accept the prosthetic condyles 122. FIG. 20 and FIG. 21 present a top plan view and a bottom plan view, respectively, of the foregoing embodiment.

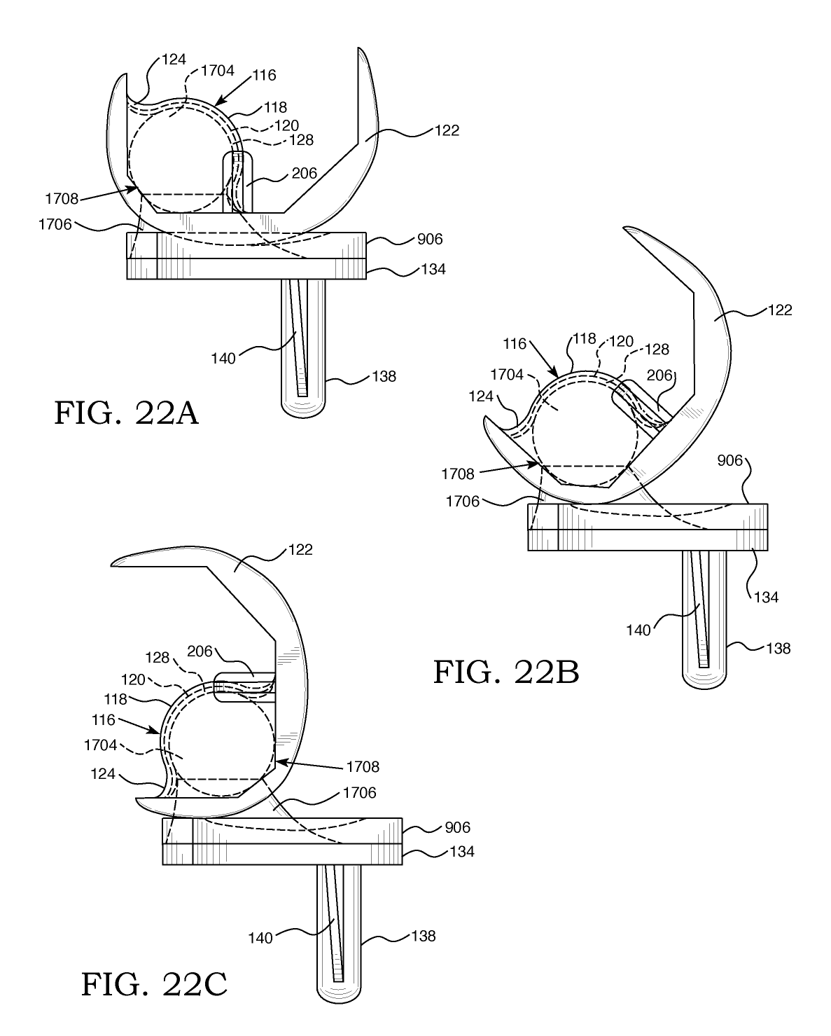

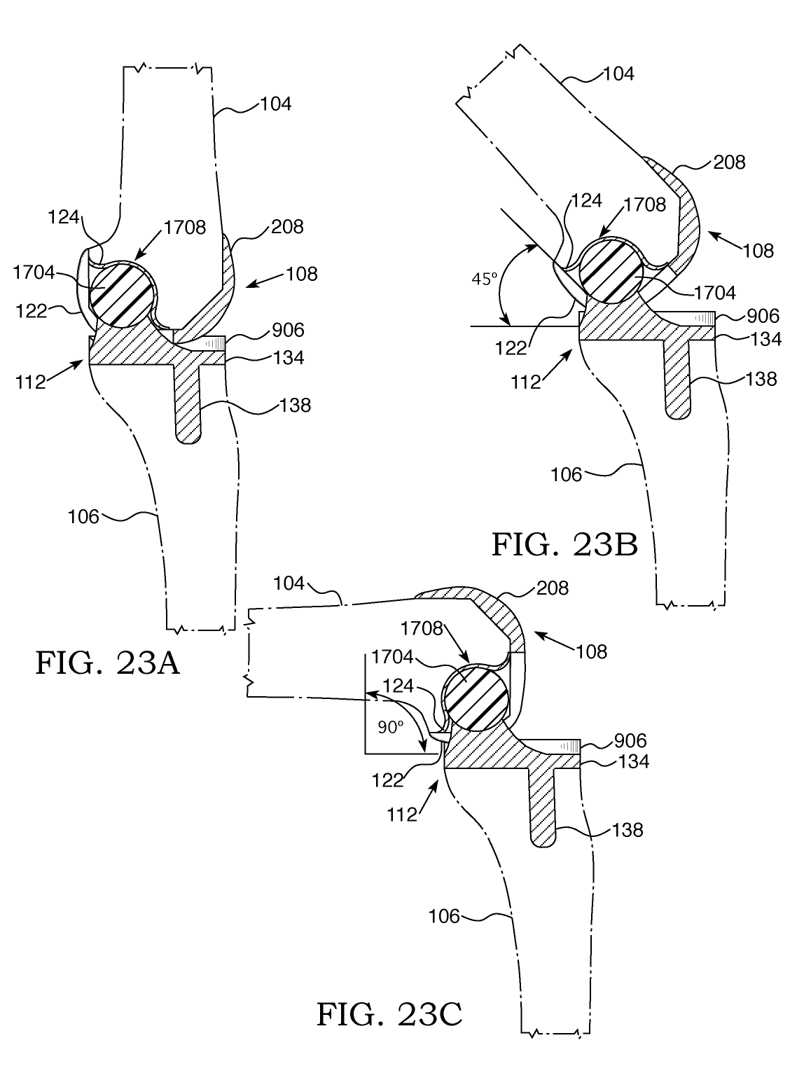

FIG. 22A, FIG. 22B, and FIG. 22C are side elevational views of an embodiment with a femoral ball 1704 integrated into a femoral cup 1708 of a femoral component 108 between prosthetic condyles 122, a tibial socket 1706 integrated into a tibial platform 134 of a tibial component 112 for receiving the tibial ball 126, and bilateral inserts 906 to accept the prosthetic condyles 122, as the prosthetic condyles 122 are positioned upon full extension of a patient's leg, a 45° flexion of a patient's leg, and a 90° flexion of a patient's leg. As therein illustrated, the femoral ball 1704 and the femoral cup 1708 together move about the axis of rotation of a femur. FIG. 23A, FIG. 23B, and FIG. 23C are cross-sectional views of this embodiment as so-configured to allow movement progressively about the center of the axis of rotation of a femur from full extension to 90° of flexion

An embodiment may employ a tibial component 112 with a tibial stein 2402 configured both to affix said tibial component 112 to the upper extremity of a tibia. A tibial stein 2402 may be configured to be larger than a prepared tibial cavity, and may be press-fit, interfacing directly with the compact bone in the tibia, creating an interference fit with the tibia or three-point fixation in the sagittal plane.

FIG. 24 is a cutaway orthogonal view of the aforesaid embodiment with a tibial component 112 having a tibial stein 2402 and an integrated trunnion 2404 with one of two reaming slots 2406 exposed. Reaming slots 2406 may be configured to allow a physician to remove bony infiltration about a tibial stein 2402 for removal of a tibial component 112 if necessary, and as illustrated in FIG. 24, may be adapted to extend from the outer surface of a trunnion and through a tibial platform into the outer surface of a tibial stein 2402.

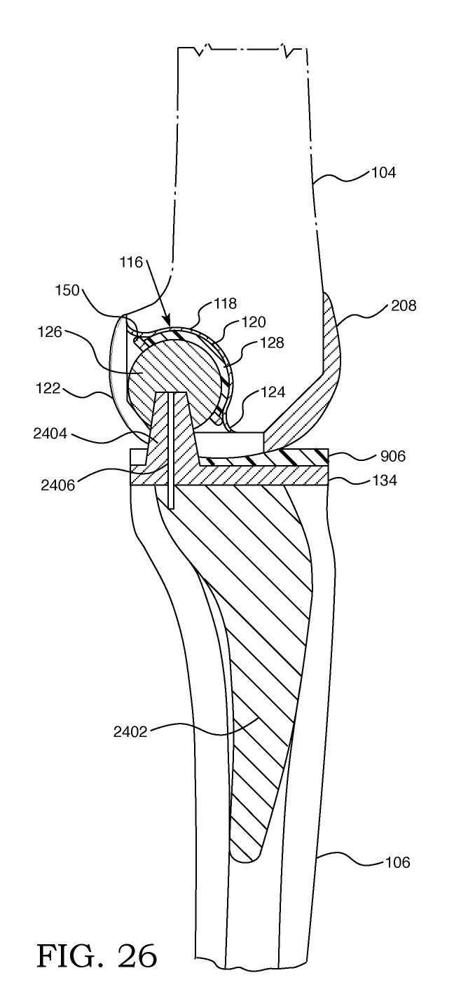

FIG. 25 is a cutaway anterior view of the aforesaid embodiment. FIG. 26 presents a cross-sectional view of an embodiment of a system 2408 illustrating a femoral component 108, a tibial component 112 with an integrated tibial trunnion 2404, a tibial ball 126 that has been received by the integrated tibial trunnion 2404, and a tibial stein 2402 integrated into said tibial component 112.

In the foregoing specification, the disclosure has been described with reference to specific exemplary embodiments thereof. The specification and drawings are, accordingly, to be regarded in an illustrative sense rather than a restrictive sense.

I claim:

1. A system comprising:
a femoral component configured to be accepted by a prepared surface of the end of a femur at its lower extremity, said femoral component having an integrated femoral socket located between prosthetic condyles of the femoral component, the femoral socket having a femoral socket liner secured to an inner surface and configured to accept a tibial ball, said femoral socket liner providing an articulating surface for said tibial ball, which tibial ball is configured to engage a modular trunnion adapted to be inserted into a trunnion post of a tibial component; and
a tibial component configured to be accepted by a prepared surface of the end of a tibia at its upper extremity, said tibial component having a trunnion post adapted to be inserted into a trunnion posthole in the end of the upper extremity of a tibia, and having one or more inserts to provide one or more articulating surfaces for said prosthetic condyles,

2. A system comprising:
a femoral component configured to be accepted by a prepared surface of the end of a femur at its lower extremity, said femoral component having an integrated femoral socket located between prosthetic condyles of the femoral component, the femoral socket having a femoral socket liner secured to an inner surface and configured to accept a tibial ball, said femoral socket liner providing an articulating surface for said tibial ball, which tibial ball is configured to engage a tibial trunnion integrated into a tibial component; and
a tibial component configured to be accepted by a prepared surface of the end of a tibia at its upper extremity, said tibial component having an integrated tibial trunnion adapted to receive a tibial ball, and having one or more inserts to provide one or more articulating surfaces for said prosthetic condyles.

3. The system of claim 2, in which said tibial component comprises a tibial stem.

4. The system of claim 3, in which the tibial stem is press-fit.

5. The system of claim 3, in which one or more reaming slots are included.

6. A system comprising:
a tibial component configured to be accepted by the prepared surface of the end of a tibia at its upper extremity, said tibial component having an integrated trunnion and ball and having one or more inserts to provide one or more articulating surfaces for said prosthetic condyles; and
a femoral component configured to be accepted by the prepared surface of the end of a femur at its lower extremity, said femoral component having an integrated femoral socket located between prosthetic condyles, the femoral socket having a femoral socket liner secured to an inner surface of the femoral socket and configured to accept a ball of an integrated trunnion and ball, said femoral socket liner providing an articulating surface for the ball of the integrated trunnion and ball.

7. The system of claim 6, in which said tibial component comprises a tibial stem.

8. The system of claim 7, in which the tibial stem is press-fit.

9. The system of claim 7, in which one or more reaming slots are included.

10. A system comprising:
a femoral component configured to be accepted by the prepared surface of the end of a femur at its lower extremity, said femoral component having a femoral ball seated between prosthetic condyles and configured to be received by a tibial socket with a tibial socket liner, said femoral ball adapted to articulate against the tibial socket liner secured to said tibial socket; and
a tibial component configured to be accepted by the prepared surface of the end of a tibia at its upper extremity, said tibial component having tibial socket integrated into a tibial plate of said tibial component and a tibial socket liner secured to said tibial socket, and having one or more inserts to provide one or more articulating surfaces for said prosthetic condyles.

11. The system of claim 10, in which said tibial component comprises a tibial stem.

12. The system of claim 11 in which the tibial stem is press-fit.

13. The system of claim 11, in which one or more reaming slots are included.

14. The system of claim 10, in which the femoral ball is affixed inside a femoral cup integrated into the femoral component by one or more joinder elements.

15. The system of claim 10, in which the femoral ball is configured to rotate freely inside a femoral cup integrated into the femoral component by one or more joinder elements.

16. The system of claim 15, in which the prosthetic condyles include inner condylar protrusions.
